(19) United States
(12) Patent Application Publication
(10) Pub. No.: US 2023/0414753 A1
(43) Pub. Date: Dec. 28, 2023

(54) STABILIZED ANTIBODY PROTEIN SOLUTIONS

(71) Applicant: Arecor Limited, Little Chesterford (GB)

(72) Inventors: Jan JEZEK, Little Chesterford (GB); Luca BADIALI, Little Chesterford (GB); David GERRING, Little Chesterford (GB)

(21) Appl. No.: 18/243,767

(22) Filed: Sep. 8, 2023

Related U.S. Application Data
(63) Continuation of application No. 16/552,682, filed on Aug. 27, 2019.
Application No. 16/552,682 is a continuation-in-part of application No. 16/487,990, filed on Jan. 16, 2020.
(60) Provisional application No. 62/723,554, filed on Aug. 28, 2018.

(30) Foreign Application Priority Data
Feb. 24, 2017 (GB) 1703062.8

(51) Int. Cl. A61K 39/395; C07K 14/705; A61K 47/18; A61K 47/10; C07K 16/22; C07K 16/28; A61K 47/60; A61K 9/08; A61K 47/26; A61K 9/00

(52) U.S. Cl. CPC A61K 39/39591; C07K 14/70521; A61K 47/183; A61K 47/10; C07K 16/22; C07K 16/2875; A61K 47/60; A61K 9/08; A61K 47/26; A61K 9/0019; C07K 16/2827; C07K 2317/21; C07K 2319/30; A61K 2039/54

(57) ABSTRACT

There is provided, inter alia, an aqueous solution comprising an antibody protein and a stabilizing amount of (i) a chelating agent which is a multi-anion; and (ii) a C3 polyol.

### CROSS-REFERENCE TO RELATED APPLICATIONS

This application is a continuation of U.S. patent application Ser. No. 16/552,682, filed Aug. 27, 2019, which claims the benefit of U.S. Provisional Patent Application No. 62/723,554, filed Aug. 28, 2018, and a continuation-in-part of U.S. patent application Ser. No. 16/487,990, 371(c) date Jan. 16, 2020, which is a U.S. National Phase of International Application PCT/GB2018/050481, filed Feb. 23, 2018, which claims priority to United Kingdom Patent Application No. 1703062.8, filed Feb. 24, 2017, each of which is incorporated herein by reference in its entirety.

### BACKGROUND

When formulated as aqueous solutions, antibody proteins are susceptible to structural degradation during storage. The processes involved in protein degradation can be divided into physical (e.g. loss of quaternary, tertiary or secondary structure, aggregation, particle formation) and chemical (i.e. processes involving a covalent change such as deamidation, aspartate isomerization, oxidation, hydrolytic clipping etc.). Each of the degradants (e.g. soluble aggregated species, insoluble aggregated species and chemically modified variants) can impact the biological activity, toxicity or immunogenicity of the antibody protein.

Therefore, the level of all degradants has to be kept within the tight specifications that are set for each antibody protein product. The rates of the degradation processes depend on temperature and antibody proteins are generally more stable at lower temperatures. Consequently, commercial antibody products must typically be stored refrigerated. However, with increasing trend toward subcutaneous products that can be self-administered by the patient, there is a strong need to develop antibody protein products that can be used outside the cold chain, at least for a period of time, such as 2 weeks, such as 4 weeks, such as 12 weeks or more. The ability to store the product outside the cold chain often results in considerable improvement in convenience for the patient during the in-use period. Allowed excursions outside the cold chain can also significantly improve shipment logistics.

The present invention addresses the problem of instability of antibody proteins, in particular the problem of antibody protein degradation.

WO2006/0096488A2 (Pharmacia & Upjohn Company LLC) describes compositions of human IgG antibodies comprising a chelating agent, said to exhibit improved chemical and/or physical stability.

WO2013/114122A2 (Arecor Limited) describes an aqueous solution comprising an antibody protein at a concentration of at least about 10 mg/mL and an oligomer of ethyleneimine, wherein the number of repeating units of ethyleneimine (n) in the oligomer is in the range of n=2-12.

WO2010/062896A1 (Abbott Laboratories) describes compositions and methods for inhibiting fractionation of immunoglobulins comprising a lambda light chain based on the observation that iron, in the presence of histidine, results in increased fragmentation of a recombinant fully human IgG molecule containing a lambda light chain due to cleavage in the hinge region.

### SUMMARY

The present invention addresses the problem of instability of antibody proteins. In one embodiment, the invention relates to an aqueous solution comprising an antibody protein and a stabilizing mixture of (i) a chelating agent which is a multi-anion; and (ii) a C3 polyol. In one embodiment, the invention provides a method of stabilizing an antibody protein in an aqueous solution (e.g. for storage) comprising the step of adding to the solution (i) a chelating agent which is a multi-anion; and (ii) a C3 polyol.

### DETAILED DESCRIPTION

The present invention relates to the discovery that an aqueous solution of antibody protein can be stabilized by including a chelating agent which is a multi-anion, and a C3 polyol.

The term “and/or” as used herein, includes the meaning of “and”, “or” and all or any other combination of the elements connected by the term.

The term “about” as used herein, means within +/−20%, preferably within +/−15%, more preferably within +/−10%, and most preferably within +/−5% of a given value or range.

The term “substantially” refers to the qualitative condition of exhibiting total or near-total extent or degree of a characteristic or property of interest. One of ordinary skill in the art will understand that biological and chemical phenomena rarely, if ever, achieve or avoid an absolute result. The term “substantially free” means that a referenced component is absent or present at a concentration below detection measured by a selected art-accepted means, or otherwise is present at a level that those skilled in the art would consider to be negligible in the relevant context.

The term “aqueous solution”, as used herein, refers to a solution in water, preferably distilled water, deionized water, water for injection, sterile water for injection or bacteriostatic water for injection. The aqueous solutions of the invention include dissolved antibody protein, a chelating agent which is a multi-anion, and a C3 polyol, and optionally, one or more additives and/or excipients. The aqueous solutions can also include one or more components, such as additives or excipients, which are partially dissolved or undissolved. The presence of such component or components will result in a multi-phase composition, such as a suspension or an emulsion. Preferably, the aqueous solution of the invention is a homogeneous solution, as determined by eye or by light-scattering.

The term “antibody protein”, as used herein, refers to an antibody, an antibody fragment, an antibody conjugated to an active moiety, a fusion protein comprising one or more antibody fragments, such as an immunoglobulin Fc domain, or a derivative of any of the aforementioned. Examples of derivatives include conjugated derivatives e.g. an antibody or antibody fragment conjugated to another moiety. Such moieties include chemically inert polymers such as PEG. Preferred antibodies include monoclonal antibodies and polyclonal antibodies, preferably monoclonal antibodies. The monoclonal antibodies can be, for example, mammalian (e.g. murine) or avian, chimeric, for example, human/mouse or human/primate chimeras, humanized antibodies or fully human antibodies. Suitable antibodies include an immunoglobulin, such as IgG, including IgG1, IgG2, IgG3 or IgG4, IgM, IgA, such as IgA1 or IgA2, IgD, IgE or IgY. In particular embodiments, the immunoglobulin is IgG1. In other embodiments, the immunoglobulin is IgG2. In other embodiments, the immunoglobulin is IgG3. In other embodiments, the immunoglobulin is IgG4. In other embodiments, the immunoglobulin is IgG2/4.

In some embodiments, the antibody protein contains one or more modifications within an Fc region that alters one or more properties of the antibody protein, such as serum half-life, complement fixation, Fc receptor binding, and/or effector function (e.g. antigen-dependent cellular cytotoxicity).

In some embodiments, the antibody protein contains one or more modifications within an Fc region that alter serum half-life by enhancing antibody binding to FcRn. Such modifications include for example, IgGl-M252Y, S254T, and T256E; IgGl-T250Q and M428L; IgGl-H433K, N434Y; IgGl-N434A; and IgGl-T307A, E380A, N434A (numbering according to Kabat EU index numbering system), and are known in the art (see, e.g. Petkova et al., Int. Immunol. 18:1759-1769 (2006); Dall'Acqua et al., J. Immunol.169:5171-5180 (2002); Oganesyan et al., Mol. Immunol. 46:1750-1755 (2009); Dall'Acqua et al., J. Biol. Chem. 281:23514-23524 (2006), Hinton et al., J. Immunol. 176:346-356 (2006); Datta-Mannan et al., Drug Metab. Dispos. 35:86-94 (2007); Datta-Mannan et al., J. Biol. Chem. 282:1709-1717 (2007); WO2006/130834; Strohl, Curr. Op. Biotechnol. 20:685-691 (2009); Yeung et al., Immunol. 182:7663-7671 (2009), and US Appl. Publ. No. 20170342167, the contents of each of which is herein incorporated by reference in its entirety).

In some embodiments, the antibody protein contains one or more modifications within an Fc region that alters serum half-life by decreasing binding of the antibody to FcRn. Such modifications include for example, IgGl-M252Y, S254T, T256E; H433K, N434F, 436H; IgGl-I253A; and IgGl-P257I, N434H and D376V, N434H (numbering according to Kabat EU index numbering system), and are known in the art (see, e.g. Petkova et al., Int. Immunol. 18:1759-1769 (2006); Datta-Mannan et al., Drug Metab. Dispos. 35:86-94 (2007); Datta-Mannan et al., J. Biol. Chem. 282:1709-1717 (2007); Strohl, Curr. Op. Biotechnol. 20:685-691 (2009); and Vaccaro et al., Nat. Biotechnol. 23:1283-1288 (2005), and US Appl. Publ. No. 20170342167, the contents of each of which is herein incorporated by reference in its entirety).

In some embodiments, the antibody protein contains one or more modifications within an Fc region that increase antibody dependent cellular cytotoxicity (ADCC). Such modifications include for example, IgGl-S298A, E333A, K334A; IgGl-S239D, 1332E; IgGl-S239D, A330L, 1332E; IgGl-P247I, A339D or Q; IgGl-D280H, K290S with or without S298D or V; IgGl-F243L, R292P, Y300L; IgGl-F243L, R292P, Y300L, P396L; and IgGl-F243L, R292P, Y300L, V305I, P396L (numbering according to Kabat EU index numbering system), and are known in the art (see, e.g. Bruhns, Blood 113:3716-3725 (2009); Shields, J. Biol. Chem. 276:6591-6604 (2001); Lazar, PNAS 103:4005-4010 (2006); Stavenhagen, Cancer Res. 67:8882-8890 (2007); Horton, Cancer Res. 68:8049-8057 (2008); Zalevsky, Blood 113:3735-3743 (2009); Bruckheimer, Neoplasia 11:509-517 (2009); WO2006/020114; Strohl, Curr. Op. Biotechnol. 20:685-691 (2009); and WO2004/074455, the contents of each of which is herein incorporated by reference in its entirety).

In some embodiments, the antibody protein contains one or more modifications within an Fc region that decrease ADCC. Such modifications include for example, IgGl-K326W, E333S; IgG2-E333S; IgGl-N297A; IgGl-L234A, L235A; IgG2-V234A, G237A; IgG4-L235A, G237A, E318A; IgG4-S228P, L236E; IgG2-118-260; IgG4-261-447; IgG2-H268Q, V309L, A330S, A331 S; IgGl-C220S, C226S, C229S, P238S; IgGl-C226S, C229S, E233P, L234V, L235A; and IgGl-L234F, L235E, P331S (numbering according to Kabat EU index numbering system), and are known in the art (see, e.g. Idusogie et al., J. Immunol. 166:2571-2575 (2001); Sazinsky et al., PNAS 105:20167-20172 (2008); Davis et al., J. Rheumatol. 34:2204-2210 (2007); Bolt et al., Eur. J. Immunol. 23:403-411 (1993); Alegre et al., Transplantation 57:1537-1543 (1994); Xu et al., Cell Immunol. 200:16-26 (2000); Cole et al., Transplantation 68:563-571 (1999); Hutchins et al., PNAS 92:11980-11984 (1995); Reddy et al., J. Immunol. 164:1925-1933 (2000); McEarchern et al., Blood 109:1185-1192 (2007); Strohl, Curr. Op. Biotechnol. 20:685-691 (2009); Kumagai et al., J. Clin. Pharmacol. 47:1489-1497 (2007); WO1997/11971; WO2007/106585; and US 2007/0148167A1, the contents of each of which is herein incorporated by reference in its entirety).

In some embodiments, the antibody protein contains one or more modifications within an Fc region that increases complement-dependent cytotoxicity (CDC). Such modifications include for example, IgGl-S298A, E333A, K334A; IgGl-S239D, 1332E; IgGl-S239D, A330L, 1332E; IgGl-P2471, A339D or Q; IgGl-D280H, K290S with or without S298D or V; IgGl-F243L, R292P, Y300L; IgGl-F243L, R292P, Y300L, P396L; and IgGl-F243L, R292P, Y300L, V305I, P396L (numbering according to Kabat EU index numbering system), and are known in the art (see, e.g. Idusogie et al., J. Immunol. 166:2571-2575 (2001); Strohl, Curr. Op. Biotechnol. 20:685-691 (2009); and Natsume et al., Cancer Res. 68:3863-3872 (2008), the contents of each of which is herein incorporated by reference in its entirety).

In some embodiments, the antibody protein contains one or more modifications within an Fc region that increase complement-dependent cytotoxicity (CDC). Such modifications include for example, IgGl-K326A, E333A; IgGl-K326W, E333S, IgG2-E333S (numbering according to Kabat EU index numbering system), and are known in the art (see, e.g. Idusogie et al., J. Immunol. 166:2571-2575 (2001); Strohl, Curr. Op. Biotechnol. 20:685-691 (2009); and Natsume et al., Cancer Res. 68:3863-3872 (2008), the contents of each of which is herein incorporated by reference in its entirety).

Suitable antibody proteins also include single chain antibodies. Also included are proteins comprising antibody fragments including Fc, Fab, Fab2, scFv fragments and the like. In some embodiments, the antibody protein is a fusion protein comprising a fragment of an immunoglobulin molecule. In some embodiments, the antibody protein is an Fc fusion protein (e.g. etanercept, abatacept, belatacept, alefacept, aflibercept, rilonacept, or luspatercept). Also embraced are single domain antibodies including nanobodies.

In certain embodiments, the antibody is fused or conjugated to an active molecule, such as a toxin or a chelating agent capable of binding a radioactive metal ion, such as ⁹⁹Tc, ¹¹¹Ir, ¹³¹I or ⁹⁰Y (e.g. ibritumomab tiuxetan and tositumomab). In particular embodiments, the antibody is an antibody-drug conjugate (ADC) (e.g. gemtuzumab ozogamicin, brentuximab vedotin, trastuzumab emtansine, or inotuzumab ozogamicin). In such embodiments, the antibody typically functions as a targeting agent, for example, directing the active molecule to cells which display a certain cell surface protein.

Specific antibodies which can be formulated as described herein include, but are not limited to, infliximab (chimeric antibody, anti-TNFα), basiliximab (chimeric antibody, anti-IL-2), abciximab (chimeric antibody, anti-GpIIb/IIIa), daclizumab (humanized antibody, anti-IL-2), gemtuzumab (humanized antibody, anti-CD33), alemtuzumab (humanized antibody, anti-CD52), edrecolomab (murine Ig2a, anti-EpCAM), rituximab (chimeric antibody, anti-CD20), palivizumab (humanized antibody, anti-respiratory syncytial virus), trastuzumab (humanized antibody, anti-HER2/neu(erbB2) receptor), bevacizumab (humanized antibody, anti-VEGF), cetuximab (chimeric antibody, anti-EGFR), eculizumab (humanized antibody, anti-complement system protein C5), efalizumab (humanized antibody, anti-CD11a), ibritumomab (murine antibody, anti-CD20), muromonab-CD3 (murine antibody, anti-T cell CD3 receptor), natalizumab (humanized antibody, anti-α4 integrin), nimotuzumab (humanized IgG1, anti-EGF receptor), omalizumab (humanized antibody, anti-IgE), panitumumab (human antibody, anti-EGFR), ranibizumab (humanized antibody, anti-VEGF), ¹³¹I tositumomab (humanized antibody, anti-CD20), ofatumumab (human antibody, anti-CD-20), certolizumab (humanized antibody, anti-TNF-α), golimumab (human antibody, anti-TNFα) and denosumab (human antibody, anti-RANK ligand). Preferred antibodies include trastuzumab, rituximab, bevacizumab, cetuximab and ipilimumab. In one embodiment, the antibody is bevacizumab. In another embodiment, the monoclonal antibody is rituximab. In one embodiment, the antibody is not an anti-TNF-α antibody. In another embodiment, the antibody is an anti-TNF-α antibody. In a further embodiment, the monoclonal antibody is adalimumab.

Other chimeric antibodies which can be formulated as described herein include bavituximab (anti-phosphatidylserine), brentuximab (anti-CD30), siltuximab (anti-IL-6), clenoliximab (anti-CD4), galiximab (anti-CD80), gomiliximab (anti-CD23), keliximab (anti-CD4), lumiliximab (anti-CD23), priliximab (anti-CD4), teneliximab (anti-CD40), vapaliximab (anti-VAP1), ecromeximab (anti-GD3), and pagibaximab (anti-staphylococcal lipoteichoic acid).

Other humanized antibodies which can formulated as described herein include epratuzumab (anti-CD22), afutuzumab (anti-CD20), bivatuzumab mertansine (anti-CD44), cantuzumab mertansine (anti-mucin), citatuzumab bogatox (anti-TACSTD1), dacetuzumab (anti-CD40), elotuzumab (anti-CD319), etaracizumab (anti-avP3-integrin), farletuzumab (anti-FRα), inotuzumab ozogamicin (anti-CD22), labetuzumab (anti-carcinoembryonic antigen), lintuzumab (anti-CD33), milatuzumab (anti-CD74), nimotuzumab (anti-EGFR), oportuzumab monatox (anti-EpCAM), pertuzumab (anti-HER2), sibrotuzumab (anti-FAP), tacatuzumab tetraxetan (anti-alpha-fetoprotein), tigatuzumab (anti-TRAIL-2), tucotuzumab celmoleukin (anti-EpCAM), veltuzumab (anti-CD20), aselizumab (anti-CD62L), apolizumab (anti-HLA-DRB), benralizumab (anti-CD125), cedelizumab (anti-CD4), epratuzumab (anti-CD22), erlizumab (anti-CD18), fontolizumab (anti-interferon-7), mepolizumab (anti-IL5), ocrelizumab (anti-CD20), pascolizumab (anti-IL4), pexelizumab (anti-complement component 5), PRO-140 (anti-CCR5), reslizumab (anti-IL5), rontalizumab (anti-interferon-α), rovelizumab (anti-CD11, CD18), siplizumab (anti-CD2), talizumab (anti-IgE), teplizumab (anti-CD3), tocilizumab (anti-IL6R), vedolizumab (anti-α4β7-integrin), visilizumab (anti-CD3), ibalizumab (anti-CD4), tefibazumab (anti-clumping factor A), tadocizumab (anti-α11bβ3-integrin), bapineuzumab (anti-amyloid-β), solanezumab (anti-amyloid-β), tanezumab (anti-NGF), urtoxazumab (anti-E. coli Shiga-like toxin II B subunit), felvizumab (anti-respiratory syncytial virus), motavizumab (anti-respiratory syncytial virus glycoprotein F) and lebrikizumab (anti-IL13).

Additional human antibodies which can be formulated as described herein include atorolimumab (anti-Rh factor), fresolimumab (anti-TGFβ-1, -2, and -3), lerdelimumab (anti-TGFβ-2), metelimumab (anti-TGFβ-1), morolimumab (anti-Rh factor), ipilimumab (anti-CTLA-4), tremelimumab (anti-CTLA-4), bertilimumab (anti-CCL11), zanolimumab (anti-CD4), briakinumab (anti-IL12 and IL-23), canakinumab (anti-IL1β), ustekinumab (anti-IL12 and IL-23), adecatumumab (anti-EpCAM), belimumab (anti-B cell activating factor), cixutumumab anti-IGF-1 receptor), conatumumab (anti-TRAIL-R2), figitumumab (anti-IGF-1 receptor), iratumumab (anti-CD30), lexatumumab (anti-TRAIL-R2), lucatumumab (anti-CD40), mapatumumab (anti-TRAIL-R4), necitumumab (anti-EGFR), olaratumab (anti-PDGF-Ra), pritumumab (anti-vimentin), robatumumab (anti-IGF-1 receptor), votumumab (anti-tumor antigen CTAA16.88), zalutumumab (anti-EGFR), stamulumab (anti-myostatin), efungumab (anti-fungal HSP90), exbivirumab (anti-hepatitis B surface antigen), foravirumab (anti-rabies glycoprotein), libivirumab (anti-hepatitis B surface antigen), rafivirumab (anti-rabies glycoprotein), regavirumab (anti-cytomegalovirus glycoprotein B), sevirumab (anti-cytomegalovirus), tuvirumab (anti-hepatitis B virus), panobacumab (anti-Pseudomonas aeruginosa serotype IATS 011), raxibacumab (anti-anthrax toxin), ramucirumab (anti-VEGF-R2), and gantenerumab (anti-amyloid-β).

Additional antibodies which can be formulated as described herein include obinutuzumab (humanized, anti-CD20), matuzumab (humanized, anti-EGFR), reslizumab (human, anti-IL5), dupilumab (human, anti-IL-4Ra), secukinumab (human, anti-IL-17A), brodalumab (human, anti-IL-17RA), dinutuximab (human anti-GD2), daratumumab (human, anti-CD38), bezlotoxumab (human, anti-C. Difficile toxin B), obiltoxaximab (chimeric, anti-PA component of B. anthracis toxin), pavilizumab (humanized, anti-RSV F protein), atezolizumab (human, anti-PD-L1), avelumab (human, anti-PD-L1), durvalumab (human anti-PD-L1), pembrolizumab (human, anti-PD-1), nivolumab (human, anti-PD-1), idarucizumab (human Fab, anti-dabigatran), evolocumab (human, anti-PCSK9), and alirocumab (human, anti-PCSK9).

Adalimumab (human, anti-TNFα) is an additional antibody that can be formulated as described herein.

Multispecific antibodies are also envisioned to be formulated as described herein. In some embodiments, the formulated antibody is bispecific. Bispecific antibodies that can be formulated as described herein include those that bind CD3 and another antigen (e.g. blinatumomab (CD3 and CD19), and anti-CD3 MUC1 bispecific antibodies). Additional bispecific antibodies that can be formulated as described herein include for example, ABT-981 (IL-1α and IL-1β), AFM13 (CD30 and CD16A), emicizumab (activated coagulation factor IX and factor X), istiratumab (IGF-1R and ErbB3), MEDI3902, ozoralizumab (TNF alpha and HSA, RG7716 (VEGF and Ang-2), SAR156597 (IL-4 and IL-13) and vobarilizumab (anti-IL-6R).

ADCs which can be formulated as described herein include for example, gemtuzumab ozogamicin (humanized, anti-CD33), brentuximab vedotin (chimeric, anti-CD30), trastuzumab emtansine (humanized, anti-HER2), and inotuzumab ozogamicin (humanized, anti-CD22).

Fusion proteins comprising a fragment of an immunoglobulin molecule can also be formulated according to the invention. Suitable fusion proteins include proteins comprising an active protein domain fused to one or more immunoglobulin fragments, such as Fc domains. Such fusion proteins include dimeric proteins having monomeric units comprising an active protein domain, such as a soluble receptor or a receptor extracellular ligand binding domain, which is fused to an immunoglobulin Fc domain. Two Fc domains can associate via disulfide bonds to form the dimeric protein. Such fusion proteins include etanercept, abatacept and belatacept. Additional fusion proteins which can be formulated as described herein include for example, alefacept, aflibercept, rilonacept, romiplostim, eloctate, luspatercept, and alprolix.

Conjugated derivatives comprising antibodies (or one or more antibody fragments) and a chemically inert polymer such as polyethylene glycol (PEG) can also be formulated according to the invention. Such derivatives include certolizumab pegol.

The antibody protein can be isolated from natural sources or can be a recombinant protein.

In certain embodiments, the antibody protein is substantially pure, that is, the composition comprises a single antibody protein and no substantial amount of any additional protein. In preferred embodiments, the antibody protein comprises at least 99%, preferably at least 99.5% and more preferably at least about 99.9% of the total protein content of the composition. In preferred embodiments, the antibody protein is sufficiently pure for use as in a pharmaceutical composition.

The antibody protein is preferably a therapeutic antibody protein. Such an antibody protein has a desirable therapeutic or prophylactic activity and is indicated for the treatment, inhibition or prevention of a disease or medical disorder.

In one embodiment, antibody protein is a monoclonal antibody such as trastuzumab, rituximab, bevacizumab, cetuximab or ipilimumab. In another embodiment, the antibody protein is an antibody derivative in which the antibody is fused or conjugated to an active molecule such as a toxin or chelating agent (e,g. an ADC such as emtuzumab ozogamicin, brentuximab vedotin, trastuzumab emtansine, or inotuzumab ozogamicin). In another embodiment, the antibody protein is a fusion protein comprising an active protein domain fused to one or more immunoglobulin Fc fragments such as etanercept, abatacept or belatacept. In a further embodiment, the antibody is a derivative of an antibody protein and is a conjugated derivative comprising one or more antibodies or antibody fragments and a chemically inert polymer, such as polyethylene glycol (e.g. certolizumab pegol).

The antibody protein is suitably present at a concentration of about 1 mg/mL to about 300 mg/mL, such as about 10 mg/mL to about 300 mg/mL, about 1 mg/mL to about 200 mg/mL or about 10 mg/mL to about 200 mg/mL. In some embodiments, the antibody protein is present at a concentration of about 25 mg/mL to about 75 mg/mL. In some embodiments, the antibody protein is present at a concentration of about, about 80 mg/mL to about 125. In some embodiments, the antibody protein is present at a concentration of about 130 mg/mL to about 180 mg/mL. In some embodiments, the antibody protein is present at a concentration of about 190 mg/mL to about 250 mg/mL. In some embodiments, the antibody protein is present at a concentration of about 50 mg/mL. In some embodiments, the antibody protein is present at a concentration of about 75 mg/mL. In some embodiments, the antibody protein is present at a concentration of about 100 mg/mL. In some embodiments, the antibody protein is present at a concentration of about 125 mg/mL. In some embodiments, the antibody protein is present at a concentration of about 150 mg/mL. In some embodiments, the antibody protein is present at a concentration of about 160 mg/mL. In some embodiments, the antibody protein is present at a concentration of about 175 mg/mL. In some embodiments, the antibody protein is present at a concentration of about 200 mg/mL. In some embodiments, the antibody protein is present at a concentration of about 225 mg/mL. In some embodiments, the antibody protein is present at a concentration of about 240 mg/mL.

The aqueous solution of the present invention comprises a chelating agent which is a multi-anion, as a stabilizing agent. By “multi-anion” is meant a species which has at least two anionic centres per molecule, at the particular pH of the solution. By “chelating agent” is meant an agent capable of complexing with metal ions such as calcium, magnesium, iron and/or zinc ions. Suitably, the chelating agent is capable of complexing with zinc ions. Typically, the multi-anion will have at least two anionic centres per molecule wherein the pH of the solution is between about pH 4.0 and about pH 8.0. In one embodiment, the chelating agent which is a multi-anion is ethylenediaminetetraacetate (EDTA). EDTA anion is preferably introduced into the aqueous solution in the form of a salt of ethylenediaminetetraacetatic acid, such as disodium or tetrasodium salt. Alternatively, it can be introduced in the form of ethylenediaminetetraacetatic acid with subsequent adjustment of pH to the required level. Further examples of a chelating agent which is a multi-anion include other chelating ions with four ionic centres such as ethylene glycol-bis (β-aminoethyl ether)-N,N,N′,N′-tetraacetate (EGTA) and 1,2-bis(o-aminophenoxy)ethane-N,N,N′,N′-tetra-acetate (BAPTA) as well as citrate, pyrophosphate and alginate. The chelating agent which is a multi-anion may be employed as a suitable salt form (e.g. as a sodium salt), or as an acid form which forms a multi-anion in solution. A mixture of chelating agents may be used. The chelating agent which is a multi-anion has a stabilizing effect and is typically present at a concentration of about 0.1 mM to about 50 mM, such as about 0.1 mM to about 20 mM, e.g. about 0.1 mM to about 10 mM. In some embodiments, the chelating agent is at a concentration of about 0.1 mM, about 1 mM, or about 10 mM. Suitably, the chelating agent is not citrate.

In one embodiment, the chelating agent which is a multi-anion does not comprise cationic centres. In one embodiment, the chelating agent which is a multi-anion contains only anionic centres.

In one embodiment, the chelating agent which is a multi-anion comprises four anionic centres per molecule. In one embodiment, the chelating agent which is a multi-anion is tetradentate.

In one embodiment, the chelating agent which is a multi-anion has a logK metal binding stability constant with respect to zinc ion binding of >5.5 at 25° C. e.g. has a logK with respect to zinc binding of >6, >6.5, >7, >7.5, >8, >8.5, >9, >9.5, >10, >10.5, >11, >11.5, >12, >12.5, >13, >13.5, >14 or >14.5 at 25° C. In one embodiment, the chelating agent which is a multi-anion has a logK with respect to zinc ion binding of between >5.5 and 15 at 25° C. e.g. has a logK with respect to zinc binding of between >6 and 15, between >6.5 and 15, between >7 and 15, between >7.5 and 15, between >8 and 15, between >8.5 and 15, between >9 and 15, between >9.5 and 15, between >10 and 15, between >10.5 and 15, between >11 and 15, between >11.5 and 15, between >12 and 15, between >12.5 and 15, between >13 and 15, between >13.5 and 15, between >14 and 15, or between >14.5 and 15, at 25° C. Metal binding stability constants listed in the National Institute of Standards and Technology reference database 46 (Critically Selected Stability Constants of Metal Complexes) can be used. The database typically lists logK constants determined at 25° C. e.g. citrate (logK=4.93), EDTA (logK=14.6), EGTA (logK=12.6), BAPTA (logK=10.26), pyrophosphate (logK=8.71) and alginate (logK=6.91). Suitably, the chelating agent which is a multi-anion has a logK with respect to zinc ion binding of between >5.5 and 15 at 25° C.

The aqueous solution of the invention also comprises a C3 polyol as a stabilizing agent which is suitably selected from 1,2-propanediol (also known as propane-1,2-diol or propylene glycol) and glycerol (also known as 1,2,3-propanetriol, glycerin or glycerine). In one embodiment, the C3 polyol is 1,2-propanediol. In another embodiment, the C3 polyol is glycerol. In a further embodiment, the C3 polyol is a mixture of 1,2-propanediol and glycerol. The C3 polyol is suitably present at a concentration of about 100 mM to about 500 mM, such as about 150 mM to about 400 mM, or about 150 mM to about 300 mM. If more than one C3 polyol is present in the aqueous solution, then the concentration refers to the total concentration of C3 polyols.

Typically, the pH of the aqueous solution of the present invention is between about pH 4.0 and about pH 8.0, such as between about pH 5.0 and about pH 7.0 or between about pH 5.0 and about pH 6.5.

In one embodiment, the aqueous solution of the invention further comprises a buffer in order to stabilise the pH of the formulation, which can also be selected to enhance antibody protein stability. Suitably the buffer is selected from the group consisting of histidine, succinate, maleate, acetate, phosphate and TRIS. In an embodiment, the buffer is phosphate buffer.

In one embodiment, a buffer is selected to have a pKa close to the pH of the composition; for example, histidine is suitably employed as a buffer when the pH of the composition is in the range 5.0-7.0. As another example, phosphate is suitably employed as a buffer when the pH of the composition is in the range 6.1-8.1. Alternatively, in another embodiment, the solution of the invention is further stabilised as disclosed in WO2008/084237A2, which describes a formulation comprising a protein and one or more additives, characterised in that the system is substantially free of a conventional buffer, i.e. a compound with an ionisable group having a pKa within 1 unit of the pH of the formulation at the intended temperature range of storage of the composition, such as 25° C. In this embodiment, the pH of the formulation is set to a value at which the formulation has maximum measurable stability with respect to pH; the one or more additives (displaced buffers) are capable of exchanging protons with the insulin compound and have pKa values at least 1 unit more or less than the pH of the formulation at the intended temperature range of storage of the formulation. The additives may have ionisable groups having pKa between 1 to 5 pH units of the pH of the formulation at the intended temperature range of storage of the composition (e.g. 25° C.). Preferably the additives have ionisable groups having pKa between 1 to 3 pH units, most preferably from 1.5 to 2.5 pH units, of the pH of the aqueous formulation at the intended temperature range of storage of the composition (e.g. 25° C.). Such additives may typically be employed at a concentration of about 0.5 mM to about 10 mM, e.g. about 2 mM to about 5 mM. In some embodiments, that additives are at a concentration of about 5 mM, about 8 mM, or about 10 mM.

Typically, the buffer is present at a concentration of about 0.5 mM to about 50 mM, such as about 1 mM to about 20 mM, e.g. about 2 mM to about 5 mM.

The aqueous solutions of the invention may optionally comprise a surfactant. In one embodiment, the surfactant is a non-ionic surfactant such as an alkyl glycoside e.g. dodecyl maltoside; a polysorbate surfactant such as polysorbate 80 or polysorbate 20; an alkyl ether of polyethylene glycol e.g. selected from polyethylene glycol (2) dodecyl ether, polyethylene glycol (2) oleyl ether and polyethylene glycol (2) hexadecyl ether; a block copolymer of polyethylene glycol and polypropylene glycol, such as poloxamer 188, poloxamer 407, poloxamer 171 or poloxamer 185; or an alkylphenyl ether of polyethylene glycol, such as 4-(1,1,3,3-tetramethylbutyl)phenyl-polyethylene glycol. Suitably the non-ionic surfactant is present at a concentration of about 10 μg/mL to about 2000 μg/mL, such as about 50 μg/mL to about 1000 μg/mL, e.g. about 100 μg/mL to about 500 μg/mL.

The aqueous solution of the invention may cover a wide range of osmolarity, including hypotonic, isotonic and hypertonic aqueous solutions. Suitably, the aqueous solution of the invention is substantially isotonic. In one embodiment, the aqueous solution of the invention is isotonic. Suitably, the osmolarity of the aqueous solution is selected to minimize pain according to the route of administration e.g. upon injection. Preferred aqueous solutions have an osmolarity in the range of about 200 mOsm/L to about 500 mOsm/L. Preferably, the osmolarity is in the range of about 250 mOsm/L to about 350 mOsm/L. More preferably, the osmolarity is about 300 mOsm/L.

Tonicity of the aqueous solution may be adjusted with a tonicity modifier. Tonicity modifiers may be charged or uncharged.

Examples of charged tonicity modifiers include salts such as a combination of sodium, potassium, magnesium or calcium ions, with chloride, sulfate, carbonate, sulfite, nitrate, lactate, succinate, acetate or maleate ions (especially sodium chloride or sodium sulphate, particularly sodium chloride). Amino acids such as glycine, histidine or arginine may also be used for this purpose. In one embodiment, the charged tonicity modifier is selected from the group consisting of sodium chloride, sodium sulphate, sodium acetate, sodium lactate, glycine, histidine and arginine. Such a charged tonicity modifier is typically present at a concentration of about 25 mM to about 500 mM, such as about 50 mM to about 250 mM, e.g. about 150 mM.

Examples of uncharged tonicity modifiers include sugars, sugar alcohols and other polyols, such as sucrose, trehalose, mannitol, raffinose, lactose, dextrose, sorbitol or lactitol, or polyethylene glycols such as PEG300 or PEG400. In one embodiment, the uncharged tonicity modifier is sucrose, trehalose, mannitol, sorbitol, PEG300 or PEG400. The C3 polyol which is a required component of the aqueous solution of the invention may function as an uncharged tonicity modifier. However, reference to an aqueous solution of the invention “further” comprising an uncharged tonicity modifier is intended to refer to an additional, further component to be added to the solution. Thus, the aqueous solution may further comprise an uncharged tonicity modifier which is other than a C3 polyol, and in particular is other than 1,2-propanediol and glycerol. Such an uncharged tonicity modifier is typically present at a concentration of about 50 mM to about 1000 mM, such as about 100 mM to about 500 mM, e.g. about 300 mM.

The aqueous solution of the invention can optionally include a preservative, suitably selected from phenol, m-cresol, chlorocresol, benzyl alcohol, propylparaben, methylparaben, benzalkonium chloride and benzethonium chloride. When present, the preservative is at a concentration of about 0.01 mM to about 100 mM. A preservative selected from phenol, m-cresol, chlorocresol, benzyl alcohol, propylparaben, methylparaben may, for example, be present at a concentration of about 10 mM to about 100 mM, such as about 20 mM to about 80 mM e.g. about 25 mM to about 50 mM. A preservative selected from benzalkonium chloride and benzethonium chloride may, for example, be present at a concentration of about 0.01 mM to about 1 mM such as about 0.05 mM to about 0.5 mM e.g. about 0.05 mM to about 0.2 mM.

In some embodiments, the disclosure provides:
[1] an aqueous solution comprising an antibody protein and a stabilizing amount of (i) a chelating agent which is a multi-anion; and (ii) a C3 polyol;
[2] a method of stabilizing an antibody protein in an aqueous solution to storage comprising the step of adding to the solution a mixture of (i) a chelating agent which is a multi-anion; and (ii) a C3 polyol;
[3] use of a mixture of (i) a chelating agent which is a multi-anion; and (ii) a C3 polyol, for stabilizing an antibody protein in an aqueous solution to storage;
[4] the aqueous solution of [1], method of [2], or use of [3], wherein the chelating agent is a chelating ion with four ionic centres;
[5] the aqueous solution, method or use of any one of [1] to [4], wherein the chelating agent which is a multi-anion is EDTA;
[6] the aqueous solution, method or use of any one of [1] to [5], wherein the chelating agent which is a multi-anion is present at a concentration of about 0.1 mM to about 50 mM, such as about 0.1 mM to about 20 mM, e.g. about 0.1 mM to about 10 mM;
[7] the aqueous solution, method or use of any one of [1] to [6], wherein the C3 polyol is 1,2-propanediol;
[8] the aqueous solution, method or use of any one of [1] to [6], wherein the C3 polyol is glycerol;
[9] the aqueous solution, method or use of any one of [1] to [6], wherein the C3 polyol is a mixture of 1,2-propanediol and glycerol;
[10] the aqueous solution, method or use of any one of [1] to [9], wherein the C3 polyol is present at a concentration of about 100 mM to about 500 mM, such as about 150 mM to about 400 mM, or about 150 mM to about 300 mM;
[11] the aqueous solution, method or use of any one of [1] to [10], wherein the antibody protein is a therapeutic antibody protein;
[12] the aqueous solution, method or use of any one of [1] to [11], wherein the antibody protein is an antibody, an antibody fragment, an antibody conjugated to an active moiety, a fusion protein comprising one or more antibody fragments, or a derivative of any of the aforementioned;
[13] the aqueous solution, method or use of [12], wherein the antibody protein is a monoclonal antibody;
[14] the aqueous solution, method or use of [13], wherein the monoclonal antibody is a murine antibody, a chimeric antibody, a humanized antibody or a human antibody;
[15] the aqueous solution, method or use of [13], wherein the monoclonal antibody is selected from trastuzumab, rituximab, bevacizumab, cetuximab and ipilimumab;
[16] the aqueous solution, method or use of [13], wherein the monoclonal antibody is rituximab or bevacizumab, or adalimumab;
[17] the aqueous solution, method or use of [12], wherein the antibody protein is a fusion protein comprising an active protein domain fused to one or more immunoglobulin Fc fragments;
[18] the aqueous solution, method or use of [12], wherein the antibody protein is etanercept, abatacept or belatacept;
[19] the aqueous solution, method or use of [12], wherein the derivative is a conjugated derivative comprising one or more antibodies or antibody fragments and a chemically inert polymer;
[20] the aqueous solution, method or use of [19], wherein the conjugated derivative is a certolizumab pegol;
[21] the aqueous solution, method or use of any one of [1] to [20], wherein the antibody protein is present at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or about 50 mg/mL, about 100 mg/mL, about 150 mg/mL, or about 200 mg/mL), optionally, wherein the antibody protein is rituximab or bevacizumab, or adalimumab;
[22] the aqueous solution, method or use of any one of [1] to [21], wherein the pH of the solution is between about pH 4.0 and about pH 8.0, such as between about pH 5.0 and about pH 7.0 or between about pH 5.0 and about pH 6.5;
[23] the aqueous solution, method or use of any one of [1] to [22], further comprising a buffer;
[24] the aqueous solution, method or use of [23], wherein the buffer is selected from the group consisting of histidine, succinate, maleate, acetate, phosphate and TRIS;
[25] the aqueous solution, method or use of [23] or [24], wherein the buffer is present at a concentration of about 0.5 mM to about 50 mM, such as about 1 mM to about 20 mM, e.g. about 2 mM to about 5 mM;
[26] the aqueous solution, method or use of any one of [1] to [25], further comprising a non-ionic surfactant;
[27] the aqueous solution, method or use of [26], wherein the non-ionic surfactant is an alkyl glycoside, such as dodecyl maltoside;
[28] the aqueous solution, method or use of [26], wherein the non-ionic surfactant is a polysorbate surfactant, such as polysorbate 80 or polysorbate 20;
[29] the aqueous solution, method or use of [26], wherein the non-ionic surfactant is an alkyl ether of polyethylene glycol;
[30] the aqueous solution, method or use of [29], wherein the alkyl ether of polyethylene glycol is selected from polyethylene glycol (2) dodecyl ether, polyethylene glycol (2) oleyl ether and polyethylene glycol (2) hexadecyl ether;
[31] the aqueous solution, method or use of [26], wherein the non-ionic surfactant is a block copolymer of polyethylene glycol and polypropylene glycol, such as poloxamer 188, poloxamer 407, poloxamer 171 or poloxamer 185;
[32] the aqueous solution, method or use of [26], wherein the non-ionic surfactant is an alkylphenyl ether of polyethylene glycol, such as 4-(1,1,3,3-tetramethylbutyl)phenyl-polyethylene glycol;
[33] the aqueous solution, method or use of any one of [26] to [32], wherein the non-ionic surfactant is present at a concentration of about 10 μg/mL to about 2000 μg/mL, such as about 50 μg/mL to about 1000 μg/mL, e.g. about 100 μg/mL to about 500 μg/mL;
[34] the aqueous solution, method or use of any one of [1] to [33], further comprising an uncharged tonicity modifier, such as sucrose, trehalose, mannitol, sorbitol, PEG300 or PEG400;
[35] the aqueous solution, method or use of [34], wherein the uncharged tonicity modifier is present at a concentration of about 50 mM to about 1000 mM, such as about 100 mM to about 500 mM, e.g. about 300 mM;
[36] the aqueous solution, method or use of any one of [1] to [35], further comprising a charged tonicity modifier, such as selected from the group consisting of sodium chloride, sodium sulphate, sodium acetate, sodium lactate, glycine, histidine and arginine;
[37] the aqueous solution, method or use of [36], wherein the charged tonicity modifier is present at a concentration of about 25 mM to about 500 mM, such as about 50 mM to about 250 mM, e.g. about 150 mM;
[38] the aqueous solution, method or use of any one of [1] to [37], wherein the aqueous solution is isotonic;
[39] the aqueous solution, method or use of any of [1] to [38], further comprising a preservative;
[40] the aqueous solution, method or use of [39], wherein the preservative is selected from the group consisting of phenol, m-cresol, chlorocresol, benzyl alcohol, propylparaben, methylparaben, benzalkonium chloride and benzethonium chloride;
[41] the aqueous solution, method or use of [39] or [40], wherein the preservative is present at a concentration of about 0.01 mM to about 100 mM;
[42] the method of [2], or of any one of [4] to [41], wherein the method for stabilizing the antibody protein is a method for inhibiting formation of high molecular weight species of the antibody protein during storage;
[43] the method of [2], or of any one of [4] to [41], wherein the method of stabilizing the antibody protein is a method for inhibiting formation of related species of the antibody protein during storage;
[44] the method of [2], or of any one of [4] to [41], wherein the method of stabilizing the antibody protein is a method for inhibiting deamidation of the antibody protein during storage;
[45] the method of [2], or of any one of [4] to [41], wherein the method of stabilizing the antibody protein is a method for inhibiting formation of low molecular weight degradation products in the aqueous solution during storage;
[46] the method of [2], or of any one of [4] to [41], wherein the method of stabilizing the antibody protein is a method for inhibiting formation of visible particles in a composition of the antibody protein during storage;
[47] the use of any one of [3] to [41], for inhibiting formation of high molecular weight species of the antibody protein during storage;
[48] the use of any one of [3] to [41], for inhibiting formation of related species of the antibody protein during storage;
[49] the use of any one of [3] to [41], for inhibiting deamidation of the antibody protein during storage;
[50] the use of any one of [3] to [41], for inhibiting formation of low molecular weight degradation products of the antibody protein during storage;
[51] the use of any one of [3] to [41], for inhibiting formation of visible particles in a composition of the antibody protein during storage; and
[52] the aqueous solution of [1] or any one of [4] to [41], wherein the solution is for administration by subcutaneous or intramuscular injection or by intravenous injection or infusion.

In some embodiments, the aqueous solution of the invention comprises,
(a) an antibody protein (e.g. a type of, or specific therapeutic antibody protein described herein such as a monoclonal antibody, multispecific antibody (e.g. a bispecific antibody), ADC, conjugated antibody derivative, or a fusion protein such as an Fc fusion protein, at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5); optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) an IgG1 antibody (e.g. trastuzumab, rituximab, bevacizumab, cetuximab, adalimumab, infliximab, ipilimumab, avelumab, alirocumab, necitumumab, obinutuzumab, ofatumumab, olaratumab, palivizumab, ramucirumab, raxibacumab, sarilumab, secukinumab, or ustekinumab) at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or about 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5); optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) an IgG2 antibody (e.g. evolocumab, eculizumab, panitumumab, denosumab, or brodalumab) at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM); and
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5); optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) an IgG4 antibody (e.g. pembroluzumab, nivolumab or dupilumab) at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or about 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5); optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) adalimumab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 110 mg/mL to about 250 mg/mL, about 110 mg/mL to about 200 mg/mL, about 120 mg/mL to about 190 mg/ml, about 130 mg/mL to about 180 mg/mL, about 140 mg/mL to about 180 mg/ml, about 150 mg/mL to about 250 mg/mL, or about 190 mg/mL to about 250 mg/mL, or about 50 mg/mL, about 100 mg/mL, about 150 mg/mL, about 160 mg/mL, about 200 mg/mL, or about 240 mg/mL);
(b) 1,2-propanediol or glycerol, or a mixture thereof at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5); optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.

In some embodiments, the aqueous solution of the invention comprises,
(a) adalimumab at a concentration of about 110 mg/mL to about 250 mg/mL, e.g. about 110 mg/mL to about 200 mg/mL about 150 mg/mL to about 250 mg/mL, about 120 mg/mL to about 190 mg/ml, or about 140 mg/mL to about 180 mg/ml, or e.g. about 50 mg/mL, about 100 mg/mL, about 150 mg/mL, about 160 mg/mL, about 200 mg/mL, or about 240 mg/mL;
(b) 1,2-propanediol or glycerol, or a mixture thereof at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
(i) optionally 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) adalimumab at a concentration of about 10 mg/mL to about 250 mg/mL, about 25 mg/mL to about 75 mg/mL, about 80 mg/mL to about 125, about 130 mg/mL to about 180 mg/mL, or about 190 mg/mL to about 250 mg/mL, or about 50 mg/mL, about 100 mg/mL, about 150 mg/mL, about 160 mg/mL, about 200 mg/mL, or about 240 mg/mL;
(b) 1, 2-propanediol or glycerol, or a mixture thereof, at a concentration of about 100 mM to about 500 mM (e.g. about 100 mM to about 300 mM, or about 100 mM to about 200 mM, e.g. about 100 mg/mL, about 130 mg/mL, or about 150 mg/mL); and
(c) a chelating agent which is a multi-anion a chelating agent (e.g. EDTA or EGTA, at a concentration of about 0.1 mM to about 20 mM, or about 0.1 mM to about 10 mM, e.g. about 0.1 mM, about 1 mM, about 10 mM, or about 20 mM); and
   wherein the pH of the aqueous solution is between about pH 5.0 and about pH 6.5, e.g. about 5.2;
   wherein the aqueous solution further comprises
(i) a buffer (e.g. histidine, succinate, maleate, acetate, phosphate or TRIS) at a concentration of about 1 mM to about 20 mM about 2 mM to about 10 mM, e.g. about 8 mM, an alkyl glycoside, such as dodecyl maltoside, a polysorbate surfactant, such as polysorbate 80, or polysorbate 20, at a concentration of about 10 μg/mL to about 2000 μg/mL (e.g.
(ii) a tonicity modifier (e.g. sodium chloride or arginine) at a concentration of about 20 mM to about 250 mM, or a concentration of about 20 mM to about 40 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM,
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) trastuzumab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) rituximab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or about 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) bevacizumab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) bevacizumab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or about 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) cetuximab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or about 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) ipilimumab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or about 50 mg/mL, about 100 mg/mL, about 150 mg/mL, about 160 mg/mL, about 200 mg/mL, or about 240 mg/mL);
(b) 1,2-propanediol or glycerol, or a mixture thereof at a concentration of about 100 mM to about 500 mM (e.g. about 150 mM to about 400 mM, about 150 mM to 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) infliximab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or about 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) ranibizumab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) pembrolizumab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) nevolimumab at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) a bispecific antibody (e.g. an antibody that binds CD3 and another antigen, such as blinatumomab) at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) a fusion protein comprising an active protein domain fused to one or more immunoglobulin Fc fragments (e.g. a peptibody, abatacept, or belatacept) at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to about 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

In some embodiments, the aqueous solution of the invention comprises,
(a) etanercept at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a 50 mg/mL, about 100 mg/mL, about 150 mg/mL, about 160 mg/mL, about 200 mg/mL, or about 240 mg/mL);
(b) 1,2-propanediol or glycerol, or a mixture thereof, at a concentration of about 100 mM to about 500 mM (e.g. about 150 mM to about 400 mM, about 150 mM to 350 mM, or about 200 mM to about 300 mM, or about 200 mg/mL, about 250 mg/mL, or about 300 mg/mL); and
(c) a chelating agent which is a multi-anion a chelating agent (e.g. EDTA, EGTA, BAPTA, citrate, pyrophosphate or alginate, or a mixture thereof), at a concentration of about 0.1 mM to about 100 mM (e.g. about 0.1 mM to about 75 mM, about 0.1 mM to about 50 mM, about 0.1 mM to about 20 mM, or about 0.1 mM to about 10 mM, or about 0.1 mM, about 1 mM, about 10 mM, or about 50 mM);
   optionally, wherein the ratio (mM/mM) of chelating agent which is a multi-anion to C3 polyol is between about 1:5 and about 1:500 (e.g. between about 1:20 and about 1:200), the ratio (wt/wt) of antibody protein to chelating agent which is a multi-anion is between about 1:1 and about 500:1 (e.g. between about 10:1 and about 200:1), the ratio (wt/wt) of antibody protein to chelating agent which is a multi-anion is between about 10:1 and about 1000:1 (e.g. between about 50:1 and about 200:1), the ratio (wt/wt) of antibody protein to C3 polyol is between about 1:5 and about 200:1 (e.g. between about 1:1 and about 50:1) or between about 1:2 and about 200:1 (e.g. between about 2:1 and about 50:1); and
   wherein the pH of the aqueous solution is between about pH 4.0 and about pH 8.0 (e.g. between about pH 5.0 and about pH 7.0 or between about pH 5.0 and about pH 6.5);
   optionally wherein the aqueous solution further comprises
(i) a buffer (e.g. histidine, succinate, maleate, acetate, phosphate or TRIS) at a concentration of about 0.5 mM to about 50 mM (e.g. about 1 mM to about 20 mM, about 2 mM to about 10 mM, or about 2 mM to about 5 mM),
(ii) a non-ionic surfactant (e.g. an alkyl glycoside, such as dodecyl maltoside, a polysorbate surfactant, such as polysorbate 80, or polysorbate 20, an alkyl ether of polyethylene glycol, such as polyethylene glycol (2) dodecyl ether, polyethylene glycol (2) oleyl ether or polyethylene glycol (2) hexadecyl ether, a block copolymer of polyethylene glycol and polypropylene glycol, such as poloxamer 188, poloxamer 407, poloxamer 171, or poloxamer 185, or an alkylphenyl ether of polyethylene glycol, such as 4-(1,1,3,3-tetramethylbutyl) phenyl-polyethylene glycol, at a concentration of about 10 μg/mL to about 2000 μg/mL (e.g. about 50 μg/mL to about 1500 μg/mL, about 50 μg/mL to about 1250 μg/mL, about 50 μg/mL to about 1000 μg/mL, about 100 μg/mL to about 750 μg/mL, or about 100 μg/mL to about 500 μg/mL, or about 500 μg/mL, about 750 μg/mL, about 1000 μg/mL, about 1250 μg/mL, or about 1500 μg/mL),
(iii) a tonicity modifier (e.g. an uncharged tonicity modifier, such as sucrose, trehalose, mannitol, sorbitol, PEG300, or PEG400, at a concentration of about 50 mM to about 1000 mM, such as about 100 mM to about 500 mM), or a charged tonicity modifier such as sodium chloride, sodium sulphate, sodium acetate, sodium lactate, glycine, histidine, or arginine, at a concentration of about 25 mM to about 500 mM such as about 50 mM to about 250 mM, about 20 mM to about 40 mM, e.g. about 25 mM to about 35 mM, about 25 mM, about 30 mM, or about 35 mM (e.g. sodium chloride at concentration of about 20 mM to about 40 mM, e.g. about 25 mM to about 35 mM, about 25 mM, about 30 mM, or about 35 mM, and/or arginine at concentration of about 25 mM to about 250 mM, or about 50 mM to about 250 mM, e.g. about 50 mM to about 200 mM, about 50 mM, about 100 mM, or about 150 mM), and/or
(iv) a preservative such as phenol, m-cresol, chlorocresol, benzyl alcohol, propylparaben, methylparaben, benzalkonium chloride, or benzethonium chloride, at a concentration of about 0.01 mM to about 100 mM.
   In some embodiments, the conductivity of the aqueous solution is >2.5 mS/cm. In some embodiments, the conductivity of the aqueous solution is <2.5 mS/cm.

The present inventors have discovered that the stability of an antibody protein in an aqueous solution is improved by the addition of a mixture of (i) a chelating agent which is a multi-anion; and (ii) a C3 polyol. The addition of a chelating agent which is a multi-anion such as EDTA has been observed to enhance the stability of an antibody protein in an aqueous solution. Surprisingly, the stabilizing effect of the EDTA is further increased by the addition of a C3 polyol. Without wishing to be bound by theory it is believed that the stabilising effect of the chelating agent which is a multi-anion is due to a combination of (i) charge interactions with positively charged patches at the surface of the protein and (ii) elimination of trace metals that may catalyse degradation processes. Without wishing to be bound by theory it is believed that the additional stabilising effect of a C3 polyol is due to optimal hydrophobic and hydrogen bond interactions at the protein surface of the small polyols leading to tighter conformation and modified interfacial tension between the protein molecules, in turn leading to lower exposure of reaction sites as well as lower probability of irreversible aggregation events.

In one embodiment, the ratio (mM/mM) of chelating agent which is a multi-anion to C3 polyol is between about 1:5 and about 1:500 e.g. between about 1:20 and about 1:200.

In one embodiment, the ratio (wt/wt) of antibody protein to chelating agent which is a multi-anion is between about 1:1 and about 500:1 e.g. between about 10:1 and about 200:1. In another embodiment, the ratio (wt/wt) of antibody protein to chelating agent which is a multi-anion is between about 10:1 and about 1000:1 e.g. between about 50:1 and about 200:1.

In one embodiment, the ratio (wt/wt) of antibody protein to C3 polyol is between about 1:5 and about 200:1 e.g. between about 1:1 and about 50:1. In another embodiment, the ratio (wt/wt) of antibody protein to C3 polyol is between about 1:2 and about 200:1 e.g. between about 2:1 and about 50:1.

The addition of a mixture of chelator which is a multi-anion, and a C3 polyol to an aqueous solution of antibody protein is expected to enhance the stability of the antibody protein, e.g. as shown in Example 1. The mixture of a chelating agent which is a multi-anion and a C3 polyol is thus referred to as a stabilizing mixture.

The “stability” of an antibody protein or a “stabilizing mixture” typically refers to a reduction of antibody protein degradation during storage. In one embodiment, “stability”/“stabilizing” refers to physical stability e.g. loss of quaternary, tertiary or secondary structure, aggregation or particle formation. In another embodiment, “stability”/“stabilizing” refers to chemical stability e.g. processes involving a covalent change such as deamidation, aspartate isomerization, oxidation or hydrolytic clipping.

It is expected that the addition of a mixture of a chelating agent which is a multi-anion, and a C3 polyol, to an aqueous solution comprising an antibody protein can enhance the stability of the antibody protein and in particular reduce the rate of antibody protein aggregation, compared with the same solution lacking the chelating agent which is a multi-anion, and a C3 polyol, following storage under the same conditions for the same length of time.

The present invention thus provides a method of stabilizing an antibody protein in an aqueous solution to storage comprising the step of adding to the solution a mixture of (i) a chelating agent which is a multi-anion; and (ii) C3 polyol. Also provided is the use of a mixture of (i) a chelating agent which is a multi-anion; and (ii) a C3 polyol, for stabilizing an antibody protein in an aqueous solution to storage. All embodiments described hereinabove with reference to the aqueous solution of the invention apply equally to the method and use of the invention.

In some embodiments, the method of the invention refers to adding (i) a chelating agent which is a multi-anion; and (ii) a C3 polyol to an aqueous solution containing a protein. In some embodiments, the method of the invention refers to adding
(i) a chelating agent which is EDTA and (ii) a C3 polyol (e.g. 1,2-propanediol and glycerol) to an aqueous solution containing an antibody protein. In some embodiments, the method of the invention refers to “the step of adding to the solution a mixture of (i) a chelating agent which is a multi-anion; and (ii) a C3 polyol”. It should be understood that the chelating agent which is a multi-anion and the C3 polyol can be added to the solution at the same time, or sequentially, and in any order (i.e. “the step” may actually include multiple steps).

The disclosure provides a method for inhibiting formation of high molecular weight species of an antibody protein in aqueous solution during storage comprising, adding (i) a chelating agent which is a multi-anion (e.g. EDTA); and (ii) a C3 polyol (e.g. 1,2-propanediol and glycerol) to the aqueous solution. Also provided is a method for inhibiting formation of high molecular weight species of an antibody protein in aqueous solution during storage, comprising the step of adding to the solution a mixture of a chelating agent which is a multi-anion, and a C3 polyol.

The disclosure provides a method for inhibiting formation of visible particles in an aqueous solution of an antibody protein during storage comprising, adding (i) a chelating agent which is a multi-anion (e.g. EDTA); and (ii) a C3 polyol (e.g. 1,2-propanediol and glycerol) to the aqueous solution. Also provided is a method for inhibiting formation of visible particles in an aqueous solution of an antibody protein during storage, comprising the step of adding to the solution a mixture of a chelating agent which is a multi-anion, and a C3 polyol.

The disclosure provides a method for inhibiting formation of related species of an antibody protein in aqueous solution during storage, comprising adding (i) a chelating agent which is a multi-anion (e.g. EDTA); and (ii) a C3 polyol (e.g. 1,2-propanediol and glycerol) to the aqueous solution. Also provided is a method for inhibiting formation of related species of an antibody protein in aqueous solution during storage, comprising the step of adding to the solution a mixture of a chelating agent which is a multi-anion, and a C3 polyol.

The disclosure provides a method for inhibiting deamidation of an antibody protein in aqueous solution during storage, comprising adding (i) a chelating agent which is a multi-anion (e.g. EDTA); and (ii) a C3 polyol (e.g. 1,2-propanediol and glycerol) to the aqueous solution. Also provided is a method for inhibiting deamidation of an antibody protein in aqueous solution during storage, comprising the step of adding to the solution a mixture of a chelating agent which is a multi-anion, and a C3 polyol.

The disclosure provides a method for inhibiting the formation of low molecular weight degradation products in an aqueous solution of an antibody protein during storage, comprising adding (i) a chelating agent which is a multi-anion (e.g. EDTA); and (ii) a C3 polyol (e.g. glycerol and/or propylene glycol) to the aqueous solution. Also provided is a method for inhibiting the formation of low molecular weight degradation products in an aqueous solution of an antibody protein during storage, comprising the step of adding to the solution a mixture of a chelating agent which is a multi-anion, and a C3 polyol.

Also provided is the use of a mixture of a chelating agent which is a multi-anion, and a C3 polyol for inhibiting the formation of high molecular weight species of an antibody protein in aqueous solution during storage.

Also provided is the use of a mixture of a chelating agent which is a multi-anion, and a C3 polyol for inhibiting the formation of visible particles in an aqueous solution of an antibody protein during storage.

Also provided is the use of a mixture of a chelating agent which is a multi-anion, and a C3 polyol for inhibiting formation of related species of an antibody protein in aqueous solution during storage.

Also provided is the use of a mixture of a chelating agent which is a multi-anion, and a C3 polyol for inhibiting deamidation of an antibody protein in aqueous solution during storage.

Also provided is the use of a mixture of a chelating agent which is a multi-anion, and a C3 polyol for inhibiting formation of low molecular weight degradation products in an aqueous solution of an antibody protein during storage.

The term “high molecular weight species” as used herein, refers to any component of the antibody protein content which has an apparent molecular weight at least about double the molecular weight of the parent active antibody protein. That is, high molecular weight species are multimeric aggregates of the parent antibody protein. The multimeric aggregates may comprise the parent antibody protein molecules with considerably altered conformation or they may be an assembly of the parent protein units in the native or near-native conformation. The determination of high molecular weight species can be done using methods known in the art, including size exclusion chromatography, electrophoresis, analytical ultracentrifugation/sedimentation velocity, light scattering, dynamic light scattering, static light scattering and field flow fractionation.

The term “low molecular weight degradation products” as used herein, refers to any component of the antibody protein content which has an apparent molecular weight less than the molecular weight of the parent active antibody protein. That is, low molecular weight degradation products are fragments of the parent antibody protein. The determination of high molecular weight species can be done using methods known in the art, including size exclusion chromatography, electrophoresis, analytical ultracentrifugation/sedimentation velocity, light scattering, dynamic light scattering, static light scattering and field flow fractionation.

The term “related species” as used herein, refers to any component of the antibody protein content formed by a chemical modification of the parent antibody protein, such as deamidated species or oxidised species. Related species are suitably detected by cation-exchange chromatography, reversed-phase chromatography or capillary electrophoresis.

Suitably an aqueous solution of the invention is sufficiently stable such that it remains substantially free of visible particles after storage at 30° C. for at least one, two or three months. Visible particles are suitably detected using the 2.9.20. European Pharmacepoeia Monograph (Particulate Contamination: Visible Particles).

Suitably the aqueous solution of the invention is sufficiently stable such that the concentration of related species remains low upon extended storage.

In one embodiment, the aqueous solution of the invention retains at least 95%, e.g. at least 96%, e.g. at least 97%, e.g. at least 98%, e.g. at least 99% parent antibody protein (by weight of total antibody protein) after storage at 30° C. for one, two or three months. The percentage of antibody protein (by weight of total antibody protein) may be determined by size-exclusion chromatography, cation-exchange chromatography, reversed-phase chromatography or capillary electrophoresis.

In one embodiment, the presence of the mixture of chelating agent which is a multi-anion, and a C3 polyol limits the increase in high molecular weight antibody protein species to no more than 5% (by weight of total antibody protein) after storage at 40° C. for one month, suitably to no more than 3% and more suitably to no more than 2%. In one embodiment, the presence of a mixture of a chelating agent which is a multi-anion, and a C3 polyol limits the increase in high molecular weight antibody protein species to no more than 5% (by weight of total antibody protein) after storage at 2-8° C. for up to two years, suitably to no more than 3% and more suitably to no more than 2%. Quantitation of high molecular weight species is as percent by weight of the total antibody protein in the aqueous solution.

In one embodiment, the presence of the mixture of a chelating agent which is a multi-anion, and a C3 polyol limits the increase in high molecular weight antibody protein species by at least 10%, preferably by at least 25%, and more preferably by at least 50% compared with an aqueous solution lacking the chelating agent which is a multi-anion, and the C3 polyol but otherwise identical, following storage under the same conditions and length of time.

In one embodiment, the presence of the mixture of a chelating agent which is a multi-anion, and a C3 polyol maintains an aqueous solution of an antibody protein free of visible aggregates while formation of visible aggregates is observed in an aqueous solution lacking the mixture of the chelating agent which is a multi-anion, and a C3 polyol but otherwise identical, following storage under the same conditions and for the same length of time. Quantification of visible aggregates can be performed by turbidity or other types of light scattering measurement.

Suitably, the aqueous solution of the invention comprises no more than 5% (by weight of total protein) high molecular weight species after storage at 40° C. for at least one, two or three months. In one embodiment, the amount of high molecular weight species increases by no more than 5% (by weight of total antibody protein), preferably no more than 3%, after storage at 40° C. for at least one, two or three months. Quantitation of high molecular weight species is as percent by weight of the total antibody protein in the aqueous solution.

Suitably, the aqueous solution of the invention should exhibit an increase in high molecular weight species during storage which is at least 10% lower, preferably at least 25% lower, more preferably at least 50% lower, than an aqueous solution lacking a mixture of a chelating agent which is a multi-anion, and a C3 polyol but otherwise identical, following storage under the same conditions and length of time.

In one embodiment, the aqueous solution of the invention is a pharmaceutical composition suitable for administration of a therapeutic antibody protein to a subject in need thereof. Such compositions can be used in a method for administering the therapeutic protein to the subject.

In another embodiment, the invention provides a method for administering a therapeutic antibody protein to a subject in need thereof. The method comprises the step of administering an aqueous solution comprising an antibody protein, a chelating agent which is a multi-anion, and a C3 polyol. Preferably the composition is administered by intravenous, subcutaneous or intramuscular injection, or infusion. More preferably the composition is administered by subcutaneous injection.

In another embodiment, the invention provides a packaged pharmaceutical composition suitable for administration to a subject in need thereof. The pharmaceutical composition comprises an aqueous solution comprising an antibody protein, a chelating agent which is a multi-anion, and a C3 polyol. The pharmaceutical composition is preferably packaged in a vial suitable for introduction of a needle for removal of the solution. In one embodiment, the pharmaceutical composition is packaged in a glass vial with a rubber stopper. The packaged pharmaceutical composition can be provided as a kit, further comprising instructions for use and, optionally, a syringe suitable for intramuscular or subcutaneous administration. Alternatively, the packaged pharmaceutical composition can be provided in the form of a pre-filled disposable syringe suitable for intramuscular or subcutaneous administration. A pre-filled auto-injector device would also be suitable for intramuscular or subcutaneous administration.

The term “pharmaceutically acceptable”, as used herein, refers to components of a pharmaceutical composition which are suitable for the intended use and mode of administration to the body of a human or an animal, such as a mammal, without undue adverse consequences, such as toxicity, irritation, and allergic response and with a reasonable risk/benefit ratio.

Abbreviations
EDTA ethylenediaminetetraacetate
PEG polyethylene glycol
HMWS high molecular weight specie
SEC size exclusion chromatography
CEX cation-exchange chromatography

### EXAMPLES

### Materials

EDTA disodium salt (Mw 372 Da), 1,2-propanediol (Mw 76 Da), glycerol (Mw 92 Da), mannitol (Mw 182 Da), NaCl (Mw 58 Da), trehalose (Mw 342 Da) were obtained from Sigma Aldrich.

### Methods of Assessing Stability of an Antibody Protein

### (a) Visual Assessment

Visible particles are suitably detected using the 2.9.20. European Pharmacepoeia Monograph (Particulate Contamination: Visible Particles). The apparatus required consists of a viewing station comprising:
a matt black panel of appropriate size held in a vertical position
a non-glare white panel of appropriate size held in a vertical position next to the black panel
an adjustable lampholder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux.

Any adherent labels are removed from the container and the outside washed and dried. The container is gently swirled or inverted, ensuring that air bubbles are not introduced, and observed for about 5 s in front of the white panel. The procedure is repeated in front of the black panel. The presence of any particles is recorded.

The visual scores are ranked as follows.
Visual score 1: Clear solution, virtually free of particles
Visual score 2: ˜5 very small particles
Visual score 3: ˜10-20 very small particles
Visual score 4: 20-50 particles, including larger particles
Visual score 5: >50 particles, including larger particles

Whilst the particles in samples with visual scores 4 and 5 are clearly detectable on casual visual assessment under normal light, samples with visual score 1-3 generally appear as clear solutions on the same assessment. Samples with visual scores 1-3 are considered to be “Pass”; samples with visual score 4-5 are considered to be “Fail”.

### (b) Size Exclusion Chromatography (SEC)

The amount of high molecular weight species is measured using a 300×7.8 mm S3000 (or equivalent) size-exclusion column with a guard column. The mobile phase is potassium phosphate pH 6.5, with a flow rate of 0.4 ml/min, injection volume of 1 μl and detected at 210 and 280 nm. The results are expressed as % high molecular species (HMWS), i.e. sum of all peak areas corresponding to aggregated protein over the sum of all protein-related peaks on the chromatogram. A small time-point to time-point variability can be observed in terms of absolute values of % HMWS, for example due to repeated size-exclusion column use. However, within a given time-point the samples are tested using the column in the same condition, so the values generated within the time-point represent a very good indication of the relative stability of the protein in the aqueous solutions tested.

### (c) Cation-Exchange Chromatography (CEX)

The amount of related species is measured using a Protein-Pak Hi Res SP column. Mobile phase A is 20 mM sodium phosphate (pH 6.5); mobile phase B is 20 mM sodium phosphate+0.5 M NaCl (pH 6.0). The following gradient elution is used: 0 min—100% A, 4 min—80% A, 10 min—55% A, 12 min—0% A. Flow rate of 1.0 ml/min; injection volume is 3 μl, with UV detection at 214 nm. The results are expressed as % main peak (i.e. native protein), % acidic species and % basic species. % Related species=% acidic species+% basic species.

### Example 1

The effect of EDTA and C3 polyols on the stability of abatacept (125 mg/ml) was investigated. The effect was tested in a background solution containing sodium phosphate (5 mM) and polysorbate 80 (0.5 mg/ml). All formulations tested were adjusted to pH 6.5. Additional excipients in the formulations tested are shown in Table 1.

TABLE 1
Additional components in formulations of abatacept tested. All formulations
contained abatacept (125 mg/ml), sodium phosphate (5 mM) and
polysorbate 80 (0.5 mg/ml) and were adjusted to pH 6.5.

| | NaCl | Trehalose | 1,2-propanediol | EDTA |
|---|---|---|---|---|
| | (mM) | (mM) | (mM) | (mM) |
| Formulation 1 | 150 | 0 | 0 | 0 |
| Formulation 2 | 0 | 300 | 0 | 0 |
| Formulation 3 | 0 | 0 | 300 | 0 |
| Formulation 4 | 150 | 0 | 0 | 1 |
| Formulation 5 | 150 | 0 | 0 | 10 |
| Formulation 6 | 150 | 0 | 0 | 50 |
| Formulation 7 | 0 | 300 | 0 | 1 |
| Formulation 8 | 0 | 300 | 0 | 10 |
| Formulation 9 | 0 | 300 | 0 | 50 |
| Formulation 10 | 0 | 0 | 300 | 1 |
| Formulation 11 | 0 | 0 | 300 | 10 |
| Formulation 12 | 0 | 0 | 300 | 50 |

Stability of Formulations 1-12 (Table 1) was tested at 25° C. and 40° C. by visual assessment and size-exclusion chromatography (SEC). Results are shown in Tables 2 and 3. It was shown that in the absence of EDTA the stability of abatacept was slightly better in the presence of an uncharged tonicity modifier (trehalose or 1,2-propanediol) than in the presence of a charged tonicity modifier (NaCl). Addition of EDTA appeared to improve the stability of abatacept, both with respect to visual assessment and with respect to the formation of HMWS. The degree of improvement was greater in compositions comprising the uncharged species. Surprisingly, the degree of improvement was greater in compositions comprising a C3 polyol (1,2-propanediol) than in compositions comprising a larger polyol (trehalose). This indicates synergistic effect between EDTA and 1,2-propanediol. Whilst the highest level of EDTA tested (50 mM) resulted in the best stability with respect to high molecular weight species it also appeared to result in slightly worse visual score. This could be due to the fact that more soluble aggregates (i.e. HMWS) are converted to insoluble aggregates in the presence of high concentration of EDTA.

TABLE 2
Visual scores of abatacept Formulations 1-12 following
storage at 25° C. and 40° C.

| | T = 0 | T = 10 weeks | T = 10 weeks |
|---|---|---|---|
| | weeks | (25° C.) | (40° C.) |
| Formulation 1 | 1 | 4 | 5 |
| Formulation 2 | 1 | 3 | 4 |
| Formulation 3 | 1 | 3 | 4 |
| Formulation 4 | 1 | 2 | 4 |
| Formulation 5 | 1 | 2 | 5 |
| Formulation 6 | 1 | 3 | 5 |
| Formulation 7 | 1 | 2 | 3 |
| Formulation 8 | 1 | 2 | 3 |
| Formulation 9 | 1 | 3 | 3 |
| Formulation 10 | 1 | 1 | 2 |
| Formulation 11 | 1 | 1 | 1 |
| Formulation 12 | 1 | 1 | 2 |

Visual score 1: clear solution, virtually free of particles;
visual score 2: ~5 very small particles;
visual score 3: ~10-20 very small particles;
visual score 4: 20-50 particles, including larger particles;
visual score 5: >50 particles, including larger particles

TABLE 3
Stability of abatacept (125 mg/ml) in Formulations 1-12 assessed by SEC.
Formation of HMWS was assessed following storage at 25° C. and 40° C.

| | HMWS (%) | | |
|---|---|---|---|
| | | T = 10 weeks | T = 10 weeks |
| | T = 0 weeks | (25° C.) | (40° C.) |
| Formulation 1 | 0.61 | 3.58 | 14.73 |
| Formulation 2 | 0.60 | 3.21 | 12.72 |
| Formulation 3 | 0.55 | 3.49 | 13.15 |
| Formulation 4 | 0.59 | 3.18 | 13.78 |
| Formulation 5 | 0.60 | 3.18 | 13.26 |
| Formulation 6 | 0.54 | 3.06 | 13.00 |
| Formulation 7 | 0.62 | 2.79 | 10.26 |
| Formulation 8 | 0.58 | 2.83 | 10.44 |
| Formulation 9 | 0.61 | 2.46 | 9.99 |
| Formulation 10 | 0.60 | 1.99 | 8.97 |
| Formulation 11 | 0.60 | 1.98 | 8.29 |
| Formulation 12 | 0.57 | 2.10 | 8.13 |

### Example 2

The effect of EDTA and polyols on stability of bevacizumab (25 mg/ml) was investigated at 40° C. The effect was tested in a background solution containing sodium phosphate (5 mM) and polysorbate 20 (0.4 mg/ml). All formulations tested were adjusted to pH 6.2. Additional excipients in the formulations tested are shown in Table 4. The stability was also compared to the composition of the currently marketed bevacizumab product (Avastin®).

TABLE 4
Additional components in formulations of abatacept tested. All formulations contained
bevacizumab (25 mg/ml) and polysorbate 20 (0.5 mg/ml) and were adjusted to pH 6.2.

| | Sodium | NaCl | Trehalose | Mannitol | Glycerol | EDTA |
|---|---|---|---|---|---|---|
| | phosphate (mM) | (mM) | (mM) | (mM) | (mM) | (mM) |
| Formulation 1 = | 50.4 | | 158 | | | |
| Composition | | | | | | |
| of Avastin ® | | | | | | |
| Formulation 2 | 5 | 130 | | | | |
| Formulation 3 | 5 | | 300 | | | |
| Formulation 4 | 5 | | | 300 | | |
| Formulation 5 | 5 | | | | 300 | |
| Formulation 6 | 5 | 130 | | | | 10 |
| Formulation 7 | 5 | 130 | | | | 50 |
| Formulation 8 | 5 | | | | 300 | 10 |
| Formulation 9 | 5 | | | | 300 | 50 |

Stability of Formulations 1-9 (Table 4) was tested at 40° C. by visual assessment. Results are shown in Table 5. Formulation 1 (i.e. the composition of Avastin®) resulted in visual score 5 following 10 weeks storage at 40° C. Similarly, visual score 5 was reached in compositions containing 5 mM sodium phosphate and either charged tonicity modifier (NaCl) or an uncharged tonicity modifier (mannitol, trehalose or glycerol). Better visual score was observed in the presence of EDTA (10 or 50 mM). However, the use of EDTA in the presence of NaCl still resulted in worse visual scores than the use of EDTA in the presence of a C3 polyol (glycerol), indicating a synergistic effect between EDTA and the C3 polyol.

TABLE 5
Visual scores of bevacizumab Formulations 1-9 following storage at 40° C.

| | T = 0 weeks | T = 10 weeks (40° C.) |
|---|---|---|
| Formulation 1 | 1 | 5 |
| Formulation 2 | 1 | 5 |
| Formulation 3 | 1 | 5 |
| Formulation 4 | 1 | 5 |
| Formulation 5 | 1 | 5 |
| Formulation 6 | 1 | 3 |
| Formulation 7 | 1 | 3 |
| Formulation 8 | 1 | 1 |
| Formulation 9 | 1 | 2 |

Visual score 1: clear solution, virtually free of particles;
visual score 2: ~5 very small particles;
visual score 3: ~10-20 very small particles;
visual score 4: 20-50 particles, including larger particles;
visual score 5: >50 particles, including larger particles.

### Example 3

The effect of EDTA and C3 polyols on stability of adalimumab (50 mg/ml, 100 mg/ml, 160 mg/ml, 200 mg/ml, and 240 mg/ml) is investigated at 40° C., 25° C., and 2-8° C. The effect is tested in a background solution containing sodium acetate (8 mM), sodium chloride (30 mM), and polysorbate 20 (1 mg/ml). All formulations tested are adjusted to pH 5.2. Additional excipients in the formulations tested are shown in Table 6.

TABLE 6
Additional components in formulations of adalimumab tested. One set of
formulations (F1-F32) is conducted for each evaluated concentration of
adalimumab (50 mg/ml, 100 mg/ml, 160 mg/ml, 200 mg/ml, and 240
mg/ml). All formulations contained adalimumab (50 mg/ml, 100 mg/ml,
160 mg/ml, 200 mg/ml, or 240 mg/ml) sodium acetate (8 mM), sodium
chloride (30 mM), and polysorbate 20 (1 mg/ml) and are adjusted
to pH 5.2.

| | Glycerol | 1,2-propanediol | EDTA | EGTA | Arginine |
|---|---|---|---|---|---|
| | (mM) | (mM) | (mM) | (mM) | (mM) |
| Formulation 1 | 200 | | | | |
| Formulation 2 | | 200 | | | |
| Formulation 3 | | | 0.1 | | |
| Formulation 4 | | | 1 | | |
| Formulation 5 | | | 10 | | |
| Formulation 6 | 200 | | 0.1 | | |
| Formulation 7 | 200 | | 1 | | |
| Formulation 8 | 200 | | 10 | | |
| Formulation 9 | 200 | | 0.1 | | 50 |
| Formulation 10 | 200 | | 1 | | 100 |
| Formulation 11 | 200 | | 10 | | 150 |
| Formulation 12 | | 200 | 0.1 | | |
| Formulation 13 | | 200 | 1 | | |
| Formulation 14 | | 200 | 10 | | |
| Formulation 15 | | 200 | 0.1 | | 50 |
| Formulation 16 | | 200 | 1 | | 100 |
| Formulation 17 | | 200 | 10 | | 150 |
| Formulation 18 | | | | 0.1 | |
| Formulation 19 | | | | 1 | |
| Formulation 20 | | | | 10 | |
| Formulation 21 | 200 | | | 0.1 | |
| Formulation 22 | 200 | | | 1 | |
| Formulation 23 | 200 | | | 10 | |
| Formulation 24 | 200 | | | 0.1 | 50 |
| Formulation 25 | 200 | | | 1 | 100 |
| Formulation 26 | 200 | | | 10 | 150 |
| Formulation 27 | | 200 | | 0.1 | |
| Formulation 28 | | 200 | | 1 | |
| Formulation 29 | | 200 | | 10 | |
| Formulation 30 | | 200 | | 0.1 | 50 |
| Formulation 31 | | 200 | | 1 | 100 |
| Formulation 32 | | 200 | | 10 | 150 |

Thus, exemplary formulations prepared in this example include: aqueous solutions containing:
(a) adalimumab (50 mg/ml, 100 mg/ml, 160 mg/ml, 200 mg/ml, or 240 mg/ml), glycerol (200 mM), EDTA (0.1 mM or 1 mM), sodium acetate (8 mM), sodium chloride (30 mM), polysorbate 20 (1 mg/ml) at pH 5.2, e.g.
   (i) adalimumab (50 mg/ml), glycerol (200 mM), EDTA (0.1 mM, 1 mM, or 10 mM), sodium acetate (8 mM), sodium chloride (30 mM), polysorbate 20 (1 mg/ml) at pH 5.2;
   (ii) adalimumab (100 mg/ml), glycerol (200 mM), EDTA (0.1 mM, 1 mM, or 10 mM), sodium acetate (8 mM), sodium chloride (30 mM), polysorbate 20 (1 mg/ml) at pH 5.2;
   (iii) adalimumab (160 mg/ml), glycerol (200 mM), EDTA (0.1 mM, 1 mM, or 10 mM), sodium acetate (8 mM), sodium chloride (30 mM), polysorbate 20 (1 mg/ml) at pH 5.2;
   (iv) adalimumab (200 mg/ml), glycerol (200 mM), EDTA (0.1 mM, 1 mM, or 10 mM), sodium acetate (8 mM), sodium chloride (30 mM), polysorbate 20 (1 mg/ml) at pH 5.2; or
   (v) adalimumab (240 mg/ml), glycerol (200 mM), EDTA (0.1 mM, 1 mM, or 10 mM), sodium acetate (8 mM), sodium chloride (30 mM), polysorbate 20 (1 mg/ml) at pH 5.2;
(b) adalimumab (50 mg/ml, 100 mg/ml, 160 mg/ml, 200 mg/ml, or 240 mg/ml), glycerol (200 mM), EDTA (0.1 mM or 1 mM), arginine (50 mM, 100 mM, or 150 mM), sodium acetate (8 mM), sodium chloride (30 mM), polysorbate 20 (1 mg/ml) at pH 5.2;
(c) adalimumab (50 mg/ml, 100 mg/ml, 160 mg/ml, 200 mg/ml, or 240 mg/ml), glycerol (200 mM), EGTA (0.1 mM, 1 mM, or 10 mM), sodium acetate (8 mM), sodium chloride (30 mM), polysorbate 20 (1 mg/ml) at pH 5.2;
(d) adalimumab (50 mg/ml, 100 mg/ml, 160 mg/ml, 200 mg/ml, or 240 mg/ml), glycerol (200 mM), EGTA (0.1 mM, 1 mM, or 10 mM), arginine (50 mM, 100 mM, or 150 mM), sodium acetate (8 mM), sodium chloride (30 mM), polysorbate 20 (1 mg/ml) at pH 5.2;
(e) adalimumab (50 mg/ml, 100 mg/ml, 160 mg/ml, 200 mg/ml, or 240 mg/ml), 1,2-propanediol (200 mM), EDTA (0.1 mM, 1 mM, or 10 mM), sodium acetate (8 mM), sodium chloride (30 mM), polysorbate 20 (1 mg/ml) at pH 5.2;
(f) adalimumab (50 mg/ml, 100 mg/ml, 160 mg/ml, 200 mg/ml, or 240 mg/ml), 1,2-propanediol (200 mM), EDTA (0.1 mM, 1 mM, or 10 mM), arginine (50 mM, 100 mM, or 150 mM), sodium acetate (8 mM), sodium chloride (30 mM), polysorbate 20 (1 mg/ml) at pH 5.2;
(g) adalimumab (50 mg/ml, 100 mg/ml, 160 mg/ml, 200 mg/ml, or 240 mg/ml), 1,2-propanediol (200 mM), EGTA (0.1 mM, 1 mM, or 10 mM), sodium acetate (8 mM), sodium chloride (30 mM), polysorbate 20 (1 mg/ml) at pH 5.2; and
(h) adalimumab (50 mg/ml, 100 mg/ml, 160 mg/ml, 200 mg/ml, or 240 mg/ml), 1,2-propanediol (200 mM), EGTA (0.1 mM, 1 mM, or 10 mM), arginine (50 mM, 100 mM, or 150 mM), sodium acetate (8 mM), sodium chloride (30 mM), polysorbate 20 (1 mg/ml) at pH 5.2.

Stability of the five sets of Formulations (5X (F1-F32)) are tested at 40° C., 25° C., and 2-8° C. by visual assessment, size-exclusion chromatography (SEC) and cation-exchange chromatography (CEX) as described above. Formation of high molecular weight species (HMWS) and low molecular weight species (LMWS) is assessed by SEC following storage for 4 and 8 weeks. Cation-exchange chromatography is used to follow % deamidated species, % oxidised species and % other related species following storage for 4 and 8 weeks. Visual score 1: clear solution, virtually free of particles; visual score 2: ˜5 very small particles; visual score 3: ˜10-20 very small particles; visual score 4: 20-50 particles, including larger particles; visual score 5: >50 particles, including larger particles.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

All publications, patents, patent applications, internet sites, and accession numbers/database sequences (including both polynucleotide and polypeptide sequences) cited are herein incorporated by reference in their entirety for all purposes to the same extent as if each individual publication, patent, patent application, internet site, or accession number/database sequence were specifically and individually indicated to be so incorporated by reference.

1. An aqueous solution comprising an antibody protein and a stabilizing mixture of (i) a chelating agent which is a multi-anion; and (ii) a C3 polyol.

2. A method of stabilizing an antibody protein in an aqueous solution to storage comprising the step of adding to the solution a mixture of (i) a chelating agent which is a multi-anion; and (ii) a C3 polyol.

3. Use of a mixture of (i) a chelating agent which is a multi-anion; and (ii) a C3 polyol, for stabilizing an antibody protein in an aqueous solution to storage.

4. The aqueous solution of claim 1, method of claim 2, or use of claim 3, wherein the chelating agent is a chelating ion with four ionic centres.

5. The aqueous solution, method or use of any one of claims 1 to 4, wherein the chelating agent which is a multi-anion is EDTA.

6. The aqueous solution, method or use of any one of claims 1 to 5, wherein the chelating agent which is a multi-anion is present at a concentration of about 0.1 mM to about 50 mM, such as about 0.1 mM to about 20 mM, e.g. about 0.1 mM to about 10 mM.

7. The aqueous solution, method or use of any one of claims 1 to 6, wherein the C3 polyol is 1,2-propanediol.

8. The aqueous solution, method or use of any one of claims 1 to 6, wherein the C3 polyol is glycerol.

9. The aqueous solution, method or use of any one of claims 1 to 6, wherein the C3 polyol is a mixture of 1,2-propanediol and glycerol.

10. The aqueous solution, method or use of any one of claims 1 to 9, wherein the C3 polyol is present at a concentration of about 100 mM to about 500 mM, such as about 150 mM to about 400 mM, or about 150 mM to about 300 mM.

11. The aqueous solution, method or use of any one of claims 1 to 10, wherein the antibody protein is a therapeutic antibody protein.

12. The aqueous solution, method or use of any one of claims 1 to 11, wherein the antibody protein is an antibody, an antibody fragment, an antibody conjugated to an active moiety, a fusion protein comprising one or more antibody fragments, or a derivative of any of the aforementioned.

13. The aqueous solution, method or use of claim 12, wherein the antibody protein is a monoclonal antibody.

14. The aqueous solution, method or use of claim 13, wherein the monoclonal antibody is a murine antibody, a chimeric antibody, a humanized antibody or a human antibody.

15. The aqueous solution, method or use of claim 13, wherein the monoclonal antibody is selected from trastuzumab, rituximab, bevacizumab, cetuximab and ipilimumab.

16. The aqueous solution, method or use of claim 13, wherein the monoclonal antibody is rituximab or, bevacizumab, or adalimumab.

17. The aqueous solution, method or use of claim 12, wherein the antibody protein is a fusion protein comprising an active protein domain fused to one or more immunoglobulin Fc fragments.

18. The aqueous solution, method or use of claim 12, wherein the antibody protein is etanercept, abatacept or belatacept.

19. The aqueous solution, method or use of claim 12, wherein the derivative is a conjugated derivative comprising one or more antibodies or antibody fragments and a chemically inert polymer.

20. The aqueous solution, method or use of claim 19, wherein the conjugated derivative is a certolizumab pegol.

21. The aqueous solution, method or use of any one of claims 1 to 20, wherein the antibody protein is present at a concentration of about 1 mg/mL to about 300 mg/mL (e.g. about 10 mg/mL to about 300 mg/mL, about 1 mg/mL to about 200 mg/mL, about 10 mg/mL to about 200 mg/mL, about 25 mg/mL to about 75 mg/mL, about 80 mg/mL to about 125 mg/mL, about 130 mg/mL to about 180 mg/mL, or about 190 mg/mL to about 250 mg/mL, or a about 50 mg/mL, about 100 mg/mL, about 150 mg/mL, or about 200 mg/mL), optionally wherein the antibody protein is rituximab, bevacizumab, or adalimumab.

22. The aqueous solution, method or use of any one of claims 1 to 21, wherein the pH of the solution is between about pH 4.0 and about pH 8.0, such as between about pH 5.0 and about pH 7.0 or between about pH 5.0 and about pH 6.5.

23. The aqueous solution, method or use of any one of claims 1 to 22, further comprising a buffer.

24. The aqueous solution, method or use of claim 23, wherein the buffer is selected from the group consisting of histidine, succinate, maleate, acetate, phosphate and TRIS.

25. The aqueous solution, method or use of claim 23 or claim 24, wherein the buffer is present at a concentration of about 0.5 mM to about 50 mM, such as about 1 mM to about 20 mM, e.g. about 2 mM to about 5 mM.

26. The aqueous solution, method or use of any one of claims 1 to 25, further comprising a non-ionic surfactant.

27. The aqueous solution, method or use of claim 26, wherein the non-ionic surfactant is an alkyl glycoside, such as dodecyl maltoside.

28. The aqueous solution, method or use of claim 26, wherein the non-ionic surfactant is a polysorbate surfactant, such as polysorbate 80 or polysorbate 20.

29. The aqueous solution, method or use of claim 26, wherein the non-ionic surfactant is an alkyl ether of polyethylene glycol.

30. The aqueous solution, method or use of claim 29, wherein the alkyl ether of polyethylene glycol is selected from polyethylene glycol (2) dodecyl ether, polyethylene glycol (2) oleyl ether and polyethylene glycol (2) hexadecyl ether.

31. The aqueous solution, method or use of claim 26, wherein the non-ionic surfactant is a block copolymer of polyethylene glycol and polypropylene glycol, such as poloxamer 188, poloxamer 407, poloxamer 171 or poloxamer 185.

32. The aqueous solution, method or use of claim 26, wherein the non-ionic surfactant is an alkylphenyl ether of polyethylene glycol, such as 4-(1,1,3,3-tetramethylbutyl)phenyl-polyethylene glycol.

33. The aqueous solution, method or use of any one of claims 26 to 32, wherein the non-ionic surfactant is present at a concentration of about 10 μg/mL to about 2000 μg/mL, such as about 50 μg/mL to about 1000 μg/mL, e.g. about 100 μg/mL to about 500 μg/mL.

34. The aqueous solution, method or use of any one of claims 1 to 33, further comprising an uncharged tonicity modifier, such as sucrose, trehalose, mannitol, sorbitol, PEG300 or PEG400.

35. The aqueous solution, method or use of claim 34, wherein the uncharged tonicity modifier is present at a concentration of about 50 mM to about 1000 mM, such as about 100 mM to about 500 mM, e.g. about 300 mM.

36. The aqueous solution, method or use of any one of claims 1 to 35, further comprising a charged tonicity modifier, such as selected from the group consisting of sodium chloride, sodium sulphate, sodium acetate, sodium lactate, glycine, histidine and arginine.

37. The aqueous solution, method or use of claim 36, wherein the charged tonicity modifier is present at a concentration of about 25 mM to about 500 mM, such as about 50 mM to about 250 mM, e.g. about 150 mM.

38. The aqueous solution, method or use of any one of claims 1 to 37, wherein the aqueous solution is isotonic.

39. The aqueous solution, method or use of any of claims 1 to 38, further comprising a preservative.

40. The aqueous solution, method or use of claim 39, wherein the preservative is selected from the group consisting of phenol, m-cresol, chlorocresol, benzyl alcohol, propylparaben, methylparaben, benzalkonium chloride and benzethonium chloride.

41. The aqueous solution, method or use of claim 39 or claim 40, wherein the preservative is present at a concentration of about 0.01 mM to about 100 mM.

42. The method of claim 2, or of any one of claims 4 to 41, wherein the method for stabilizing the antibody protein is a method for inhibiting formation of high molecular weight species of the antibody protein during storage.

43. The method of claim 2, or of any one of claims 4 to 41, wherein the method of stabilizing the antibody protein is a method for inhibiting formation of related species of the antibody protein during storage.

44. The method of claim 2, or of any one of claims 4 to 41, wherein the method of stabilizing the antibody protein is a method for inhibiting deamidation of the antibody protein during storage.

45. The method of claim 2, or of any one of claims 4 to 41, wherein the method of stabilizing the antibody protein is a method for inhibiting formation of low molecular weight degradation products in the aqueous solution during storage.

46. The method of claim 2, or of any one of claims 4 to 41, wherein the method of stabilizing the antibody protein is a method for inhibiting formation of visible particles in a composition of the antibody protein during storage.

47. The use of any one of claims 3 to 41, for inhibiting formation of high molecular weight species of the antibody protein during storage.

48. The use of any one of claims 3 to 41, for inhibiting formation of related species of the antibody protein during storage.

49. The use of any one of claims 3 to 41, for inhibiting deamidation of the antibody protein during storage.

50. The use of any one of claims 3 to 41, for inhibiting formation of low molecular weight degradation products of the antibody protein during storage.

51. The use of any one of claims 3 to 41, for inhibiting formation of visible particles in a composition of the antibody protein during storage.

52. The aqueous solution of claim 1 or any one of claims 4 to 41, wherein the solution is for administration by subcutaneous or intramuscular injection or by intravenous injection or infusion.
